(19) United States
(12) Patent Application Publication
(10) Pub. No.: US 2007/0282331 A1
(43) Pub. Date: Dec. 6, 2007

(54) Bipolar high-frequency incision tool for an endoscope

(75) Inventor: Tomohiro Kawano, Saitama-ken (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/442,424

(22) Filed: May 30, 2006

(51) Int. Cl. A61B 18/14

(52) U.S. Cl. 606/48

(57) ABSTRACT

A bipolar high-frequency incision tool for an endoscope having a flexible insertion portion configured to be inserted into a body cavity. First and second elongated electrodes mounted to a distal end of the insertion portion so as to be movable between an open position and a closed position. The first and second electrodes being connected with conductive wires that provide a high frequency voltage, and having corresponding first and second surfaces. The first and second surfaces are spaced apart from each other when the first and second electrodes are in an open position, and the first and second electrodes remain spaced from one another when the first and second electrodes are moved to the closed position. In addition, the first and second surfaces each have inwardly tapered side surfaces configured to form respective blade edges.

### FIELD OF THE INVENTION

The present invention relates to a bipolar high-frequency incision tool, and more particularly relates to an incision tool for an endoscope having generally hook shaped electrodes.

### BACKGROUND AND MATERIAL INFORMATION

It is known in the art to provide a high frequency incision instrument, which utilizes a needle or generally rod-shaped electrode in combination with a large counter electrode placed on the body surface of the patient. High frequency electric current is generated between the needle or rod-shaped electrode and the counter electrode to cauterize the tissue in the vicinity of the needle or rod-shaped electrode. Further, because the high frequency incision instrument, of the rod or needle type, is monopolar there is a large heat impact to a surrounding area of tissue.

The disadvantage of the prior art high frequency incision instrument is that it may make a hole in the tissue that is much deeper than required if the high frequency current is generated continuously for a long period of time since the current flows from one electrode located inside the human body to the other electrode placed on the outer body surface.

Forming such a deep hole can be avoided by generating the current intermittently; however, this requires longer surgery.

Thus, there is a need for a bipolar high-frequency incision instrument for an endoscope that can confirm an appropriate incision to be made, during a relatively short surgery time.

### SUMMARY OF THE INVENTION

A non-limiting embodiment of the present invention provides a bipolar high-frequency incision tool having a flexible insertion portion configured to be inserted into a body cavity through an endoscope. The bipolar high-frequency incision tool has first and second elongated electrodes mounted to a distal end of an insertion portion so as to be movable between an open position and a closed position The first and second electrodes are connectable to conductive wires that provide a high frequency voltage, and have corresponding first and second inwardly facing curved surfaces, respectively. The first and second surfaces are spaced apart from each other when the first and second electrodes are in an open position, and the first and second electrodes remain laterally spaced from each other when the first and second electrodes are moved to the closed position. In addition, the first and second electrodes each may have a generally hook shape.

In another feature, the first and second electrodes each may have curved tips located at respective distal ends. In addition, the curved tips may be spaced from each other, at the distal ends of the electrodes, in a direction generally perpendicular to a longitudinal extent of the electrodes.

According to another feature, the electrodes may have a generally straight outwardly facing surface; and in a further feature, the curved tips may overlap, when in a closed position, to form a generally elliptical shape between the electrodes. In addition, the first and second electrodes are movable between the open and closed positions along offset planes.

In yet another feature, the electrodes are configured to pull a desired amount of tissue. In addition, opposing surfaces of each electrode that meet to form each curved tip define acute angles therebetween.

In a further aspect of the invention, a tool for an endoscope is provided that includes a supporting member configured to be connected to an insertion portion of the endoscope, the first and second jaws are pivotally connected to the supporting member and connectable to operating wires that are operable to move the jaws between open and closed positions, the jaws each having an inwardly facing curved surface, and wherein the first and second jaws remain laterally spaced from each other when the first and second are moved to the closed position. The first and second jaws may each have a generally hook shape, and they may each have curved tips provided at a distal end thereof. The curved tips may be spaced from each other in a direction generally parallel to a longitudinal extent of said jaws.

In other aspects, the jaws may each further include a generally straight outwardly facing surface. Also, the curved tips may overlap, when in a closed position, to form a generally elliptical shape between the jaws. Moreover, the first and second jaws are movable between said open and closed positions along offset planes, and the jaws may be configured to pull a desired amount of tissue. Additionally, opposing surfaces forming each said curved tip define an acute angle therebetween.

### BRIEF DESCRIPTION OF THE DRAWINGS

The present invention is further described in the detail description which follows, in reference to the noted plurality of drawings, by way of non-limiting examples of preferred embodiments of the present invention, in which like characters represent like elements throughout the several views of the drawings, and wherein:

FIG. 1 shows a perspective view of a bipolar high-frequency incision tool in a closed position according to an embodiment of the invention;

FIG. 2 shows a top plan view of the bipolar high-frequency incision tool of FIG. 1 shown connected to operating wires surrounded by an insulating tube and sheath;

FIG. 3 shows a cross-section of FIG. 2 taken along line A-A;

FIG. 4 shows a perspective view of an affected area and the bipolar high-frequency incision tool in an open position; and

FIG. 5 shows a perspective view of the affected area and an incision being formed by the bipolar high-frequency incision tool when in a closed potion.

### DETAILED DESCRIPTION OF THE DRAWINGS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

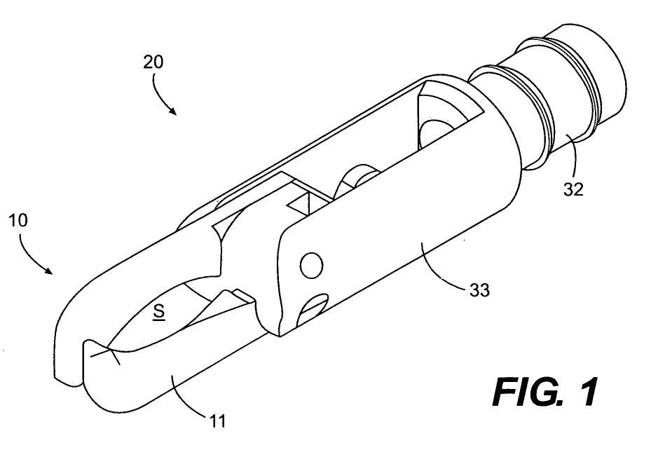

Referring to the drawings, wherein like characters represent like elements, FIG. 1 shows a perspective view of a bipolar high-frequency incision tool 10 for an endoscope according to a non-limiting embodiment of the present invention. The tool may be used in conjunction with a bipolar high-frequency endoscopic surgical system described, for example, in U.S. Pat. No. 6,969,389 and U.S. Patent Publication No. 2003/0191465, both disclosures being expressly incorporated herein by reference in their entireties.

FIG. 1 schematically shows a perspective view of a bipolar high-frequency incision tool 10 for an endoscope according to a first embodiment of the invention. The bipolar high-frequency incision tool 10 is connected to a high frequency power supply (not shown).

The bipolar high-frequency incision tool 10 includes an operation portion (not shown) and an insertion portion 20 connected to a distal end of the operation portion. 100171 The insertion portion 20 is configured to be introduced into a body cavity through a treatment tool insertion channel of an endoscope (not shown). The insertion portion 20 includes an elongated and flexible sheath 30, a pair of conductive wires 31, as shown in FIG. 3, slidably inserted through the sheath 30, and a pair of electrodes 11 provided at the distal end of the insertion portion 20 and connected to the conductive wires 31. The sheath 30 is preferably made of any suitable insulating material, e.g., poly-tetra-fluoro-ethylene (PTFE).

The conductive wires 31 may be detachably connected to a high frequency power supply (not shown). In this regard, one of the conductive wires 31 is connected to a positive terminal of the power supply and the other to the negative terminal, thereby providing power to the bipolar high-frequency incision tool 10.

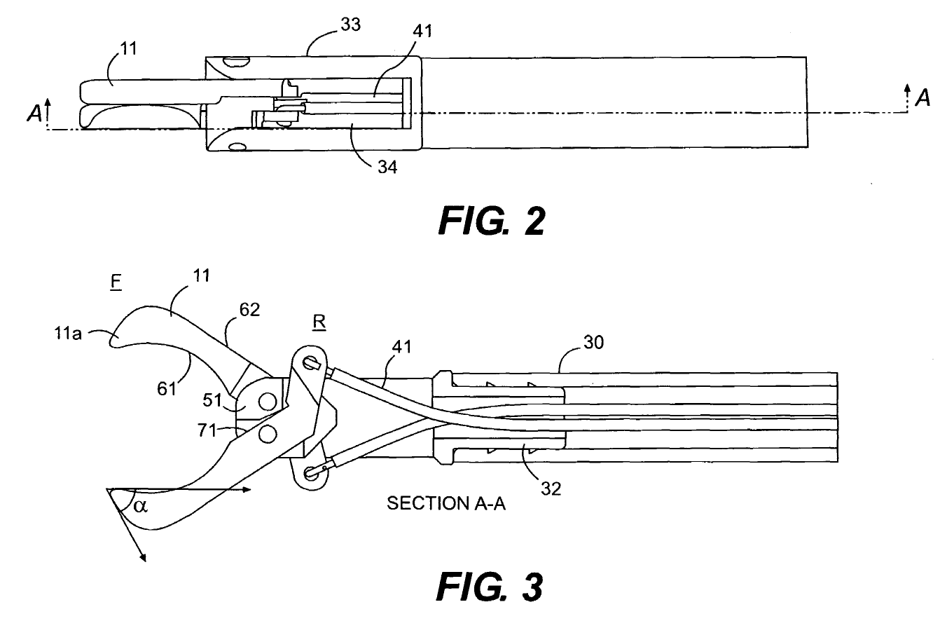

FIG. 3 shows a sectional side view of the distal end portion of the bipolar high-frequency incision tool 10 shown in FIG. 2. A pair of jaws or electrodes 11 is shown in a closed position in FIG. 1, and at an open position in FIG. 3.

As shown in FIGS. 1-3, a supporting member 32 that supports the pair of electrodes 11 is mounted to the distal end of the flexible sheath 30. The supporting member 32 may be made of any suitable hard insulating material, such as rigid synthetic resin. The supporting member 32 has two arms 33 that extend in a forward direction and parallel to each other to form a slit 34 having a generally uniform width. Two pins 35 are supported between the arms 33 in the vicinity of the distal end thereof. The pins 35 are arranged generally parallel to and spaced apart from each other, and generally perpendicular to side walls of the slit 34. The pins 35 may be made of any suitable material, such as stainless steel.

The pair of electrodes 11 is partially provided within the slit 34 of the supporting member 32 and each is rotatably mounted to a respective one of the pair of pins 35. Thus, the pair of electrodes 11 are pivotally movable between the closed position shown in FIG. 1, at which the tips of the electrodes 11 overlap but remain laterally spaced from each other by a small amount (for example about 0.05 mm to about 0.5 mm), and the open position shown in FIG. 3 in which the tips of the electrodes 11 are located spaced apart from each other.

The rear ends R of the electrodes 11 are connected with the conductive wires 31. Each of the conductive wires 31 is covered with an insulating tube 41 except the end portion thereof at which the conductive wire 31 is connected to the corresponding electrode 11.

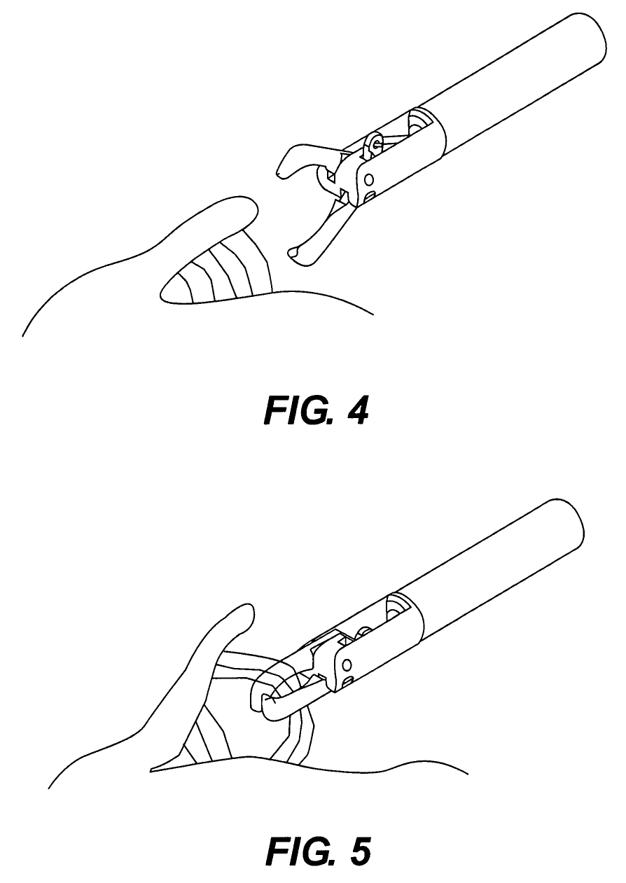

An insulating block 51, as shown in FIG. 4, is provided on the other end of the supporting member 32 to prevent the electrodes 11 from coming into contact with each other within the slit 34. The insulating block 51 is located between the electrodes 11 and may be formed in one piece with the support member 32, or may be formed separately and supported by the pins 35. The insulating block may be made of any suitable insulating material such as a resin, e.g., poly-tetra-fluoro-ethylene (PTFE).

FIG. 4 is a perspective view of the insertion portion 20. The electrodes 11 are elongated generally hook-shaped opposed members that may be made of any suitable electrode material such as metal, e.g., stainless steel. The electrodes 11 include a generally elongated front portion F and a generally elongated rear portion R. When the electrodes 11 are mounted to the supporting member 32, the front portion is located at a position forward of the arms 33, and the rear portion is positioned generally between the arms 33 (see FIG. 3).

Two through holes may be provided in the rear portion R of each electrode 11. One through hole, a supporting through hole, is configured to be a supporting hole to receive a pin 35 and is located generally at the center of each electrode 11. The other one is a connection hole for a wire 31 and is provided in the vicinity of the rear portion of each electrode 1.

As shown in FIG. 3, each electrode 11 is pivotably mounted to the supporting member 32 by insertion the corresponding pins 35 through a respective supporting hole. Thus, each electrode 11 can swing between the closed position shown in FIG. 1 and the opened position shown in FIG. 3.

As can be seen in FIG. 3, the distal end of each conductive wire 31, which is exposed from the corresponding insulating tube 41, is passed through the connecting hole to be connected to respective electrodes 11.

The rear portion R of each electrode 11 is slightly offset so that the conductive wires 31 that slide back and forth within the sheath 30 can swing the electrodes 11 around corresponding pin 35 between the open and closed positions.

The electrodes 11 have corresponding first and second inwardly facing curved surfaces 61, respectively. In addition, the electrodes each have generally straight outwardly facing surfaces 62, provided opposite the inwardly facing curved surfaces. Further, the electrodes are provided with curved tips 11a at their distal ends. Thus, it should be appreciated that the configuration of the electrodes, including the curved tips 11a, ensure the secure and safe resection of only a desired portion of an affected area; i.e., by pulling the bipolar high-frequency incision tool 10 a surgeon can confirm/detect whether an appropriate amount of tissue has been pinched (see FIG. 5).

The first and second inwardly facing curved surfaces 61 are also provided to have a generally elliptical spacing S along a length of the first and second surfaces 61 when the electrodes 11 are in a closed position, as shown in FIG. 1. In addition, the generally elliptical spacing S located between the electrodes 11 allows a desired amount of tissue to be pinched; thereby contributing to safe and secure treatment of the affected tissue.

The inwardly facing curved surfaces 61 may be provided on only a portion of the respective electrode. Therefore, a portion of each electrode located between the slightly offset ends (approximate the rear portion) and respective inwardly facing curved surfaces 61 may comprise a generally flat surface 71 that abuts the insulating block 51.

FIG. 4 shows the bipolar high-frequency incision tool 10 positioned about to form an incision in a desired area. In addition, the first and second electrodes 11 are moveable between the open and closed positions along offset plans that are slightly spaced from each other in a direction generally perpendicular to the longitudinal extent of the electrodes 11. In addition, the opposing surfaces that form the curved tips 11a, provided at respective distal ends, may further define an acute angle α therebetween (see FIG. 3). Of course, the smaller the angle α, the sharper the tips 11a will be, and the tip sharpness may be selected based on the surgery to be performed.

It is noted that those skilled in the art will readily understand that to assist the surgeon in performing any of the bipolar high-frequency incision tool 10 operations, different qualities of high-frequency voltage may be used (e.g., different duration, voltage and the like)

It is further noted that the foregoing examples have been provided merely for the purpose of explanation and are in no way to be construed as limiting of the present invention. While the present invention has been described with reference to a preferred embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A bipolar high-frequency incision tool for an endoscope, comprising:
a flexible insertion portion configured to be inserted into a body cavity through an endoscope;
first and second elongated electrodes mounted to a distal end of said insertion portion so as to be movable between an open position and a closed position, said first and second electrodes being connectable with conductive wires that provide a high frequency voltage, said first and second electrodes having corresponding first and second inwardly facing curved surfaces, respectively;
wherein said first and second inwardly facing curved surfaces are spaced apart from each other when said first and second electrodes are in an open position, and said first and second electrodes remain laterally spaced from each other when said first and second electrodes are moved to said closed position.

2. The bipolar high-frequency incision tool according to claim 1, wherein said first and second electrodes each have a generally hook shape.

3. The bipolar high-frequency incision tool according to claim 1, wherein said first and second electrodes each have curved tips provided at a distal end thereof.

4. The bipolar high-frequency incision tool according to claim 3, wherein said curved tips are spaced from each other in a direction generally parallel to a longitudinal extent of said electrodes.

5. The bipolar high-frequency incision tool according to claim 1, wherein said electrodes each further comprise a generally straight outwardly facing surface.

6. The bipolar high-frequency incision tool according to claim 3, wherein said curved tips overlap, when in a closed position, to form a generally elliptical shape between the electrodes.

7. The bipolar high-frequency incision tool according to claim 1, wherein said first and second electrodes are movable between said open and closed positions along offset planes.

8. The bipolar high-frequency incision tool according to claim 1, wherein said electrodes are configured to pull a desired amount of tissue.

9. The bipolar high-frequency incision tool according to claim 3, wherein opposing surfaces forming each said curved tip define an acute angle therebetween.

10. A tool for an endoscope, comprising:
a supporting member configured to be connected to an insertion portion of the endoscope;
first and second jaws pivotally connected to the supporting member and connectable to operating wires that are operable to move the jaws between open and closed positions, said jaws each having an inwardly facing curved surface; and
wherein said first and second jaws remain laterally spaced from each other when said first and second are moved to the closed position.

11. The tool according to claim 10, wherein said first and second jaws each have a generally hook shape.

12. The tool according to claim 10, wherein said first and second jaws each have curved tips provided at a distal end thereof.

13. The tool according to claim 12, wherein said curved tips are spaced from each other in a direction generally parallel to a longitudinal extent of said jaws.

14. The tool according to claim 10, wherein said jaws each further comprise a generally straight outwardly facing surface.

15. The tool according to claim 12, wherein said curved tips overlap, when in a closed position, to form a generally elliptical shape between the jaws.

16. The tool according to claim 1, wherein said first and second jaws are movable between said open and closed positions along offset planes.

17. The tool according to claim 10, wherein said jaws are configured to pull a desired amount of tissue.

18. The tool according to claim 12, wherein opposing surfaces forming each said curved tip define an acute angle therebetween.
